(19) United States
(12) Patent Application Publication
(10) Pub. No.: US 2019/0008440 A1
(43) Pub. Date: Jan. 10, 2019

(54) Method and device to investigate or treat painful neuropathy

(71) Applicant: Mikhail Nemenov, Mountain View, CA (US)

(72) Inventor: Mikhail Nemenov, Mountain View, CA (US)

(21) Appl. No.: 15/731,630

(22) Filed: Jul. 10, 2017

(51) Int. Cl. A61B 5/00; A61B 5/01

(52) U.S. Cl. CPC A61B 5/4041; A61B 5/0075; A61B 2562/0233; A61B 5/015; A61B 5/0077

(57) ABSTRACT

A process and laser system for in vitro and in vivo pain research, pain clinical testing and pain management. In preferred embodiments of the present invention a diode laser operating at a 980 nm wavelength is used to produce warmth, tickling, itching, touch, burning, hot pain or pin-prick pain. The device and methods can be used for stimulation of a single nerve fiber, groups of nerve fibers, nerve fibers of single type only as well as more the one type of nerve fibers simultaneously. The device and the methods can be applied in a wide variety of situations involving the study and treatment of pain. Preferred embodiments of the present invention provide laser systems and techniques that permit mapping and selective activation of silent, C-mechano-insensitive fibers or A-delta or C-polymodal fibers, de-functionalization (depleting) of these fibers for the purpose of the treatment of peripheral neuropathy and monitoring of CGRP and substance P associated with stimulation of silent, C-mechano-insensitive fibers.

### CROSS REFERENCE TO RELATED APPLICATIONS

This application is a continuation-in-part to of Provisional Application Ser. No. 60/360,324 filed Jul. 9, 2016.

### FIELD OF INVENTION

This invention relates medical instruments and processes and in particular to instruments and processes for diagnostic and management of pain.

### BACKGROUND OF THE INVENTION

Chronic pain places an enormous toll on the human society. Pain syndromes affect more than 30 million Americans per year, costing more than 100 billion dollars in medical expenses and lost work, in addition to the immeasurable expense of human suffering. A migraine headache is an intensely throbbing headache, often in only one part of the head. It may occur as often as daily or only once or twice a year. It may last for only a couple of hours or for days. However long it lasts, a migraine typically causes severe, even disabling, pain. Unfortunately, currently available means of assessing the pain patients are limited in their predictive value in terms of directing treatment regimens and its affect over 100 million Americans per year.

### Prior-Art Research and Medical Devices for Stimulation of Nociceptors

Contemporary basic pain research has a number of significant problems and serious limitations. One of these problems is the inability to assess a source of spontaneous pain in neuropathic pain and migraine patients. The problem is apparently associated with C-mechano-insensitive (CMi) fibers and the inability of the patient and/or physician to affect the activation threshold of CMi fibers and/or concentration of neuropeptides such as Substance P (SP) and calcitonin gen-related peptide (CGRP) on timely basis. The modern understanding of prevention of migraine is based on blocking by CGRP and its receptors. CGRP is believed to play critical role in inflammatory pain also. The modern understanding of management of peripheral neuropathic is based on knock out spontaneous activity of CMi fibers that are silent in healthy subjects.

CMi fibers are subtype of C-pain sensing fibers (nociceptors) that are located deep in the skin (in dermis) of animals and humans and is uniquely responsible for neurogenic flare when activated. The neurogenic or aka axon reflex flare is significant increasing of blood perfusion that is associated with release of substance P and/or CGRP. The threshold of neurogenic flare is equivalent of activation threshold of CMi fibers. Therefore, the process and device that defines a threshold of activation of CMi fibers will serve both populations of peripheral painful neuropathy and migraine patients.

In patents with ongoing neuropathic pain CMi fibers are abnormally spontaneously active and sensitized to the heat and producing spontaneous activity of C-nociceptors in painful polyneuropathy with the result that activation thresholds have been lowered. However, in healthy subjects as well as in pain patients in whom CMi are spontaneously active, the activation thresholds of CMi fibers are higher compared to pain thresholds of C polymodal fibers to heat (contact or radiant) and/or electrical stimulation. Heating of deeper skin layers might detect spontaneously active heat-sensitized nociceptors. Therefore, an activation of CMi fibers is associated with over threshold pain simulation of C polymodal fibers by any currently available stimulation and selective activation is believed to be impossible. Because, activation of CMi fibers as well as neurogenic flare is associated with induced supra-threshold pain it is unpractical for application in clinical trials, diagnostic or monitoring of CGRP and efficacy of CGRP blockers. The currently used capsaicin stimulation of neurogenic flare does not allow a repeatable and reproducible stimulation because capsaicin solution residual time is over 48 hours and duration of capsaicin induced flare is lasting over several hours.

Progress in pain and migraine research and treatment has been retarded because of the lack of adequate equipment to assess, test and treat (defunctionalize, knock out spontaneous activity) of CMi in a variety of clinical conditions and measure. On the other hand activation of CMi nerve fibers is uniquely related to release of substance P and CGRP. The suppression and control of them may prevent migraine. In order to obtain statistically proven data during the testing of pain therapy in every phase of analgesic drug or device treatment development including clinical trials, it is desirable (and may be necessary) to have objective parameters to estimate the level of pain. If pain could be quantified, this could enormously shorten the time of clinical trials and diagnostics needed to demonstrate statistically significant clinical results.

Pain stimulators currently available on the market include U.S. Pat. No. 7,402,167 granted to Applicant. Other stimulators suffer from:
low level of repeatability
inconvenient heat delivery
lack of fiber type specificity quantitative sensory effects
lack of ability to objectively estimate degree of pain
lack of ability to produce of single (mono-modal) skin pain sensations as well as other mono-modal skin sensations
lack of ability to selectively assess pain mediated by the activation of different classes of pain sensing nerve cells (or fibers)

Radiant heat stimulators are used in animal and human pain research. The main problem is that the temperature activation threshold of C polymodal fibers to radiant heat stimulation is about 39° C. and CMi fibers over 47° C. that is higher even than pain threshold of C polymodal fibers. These devices are limited by accuracy of intensity and produce relatively large spots. Heating rates are generally too low to accurately measure very short response times. Some prior art pain stimulators are described in the following patents which are incorporated herein by reference: U.S. Pat. Nos. 5,191,896, 6,248,079, and 5,025,796.

Contact heat (contact thermode) stimulators can be used as pain stimulators; however, these devices have the same limitation as radiant heat stimulators can produce substantial tissue damage in the course of producing the activation of CMi fibers. Some of these prior art pain stimulators are described in the following patents which are incorporated herein by reference: U.S. Pat. No. 4,763,666 and US Application No. 2015/0127077.

The present invention provides process and method for selective activation and/or de-functionalization (i.e. depleting) of CMi fibers for the purpose of the diagnostic, prediction and/or treatment of peripheral neuropathy and migraine and biomarker for monitoring of efficacy of the treatment of peripheral neuropathy and migraine.

### Use of Lasers

It is known to use lasers for producing pain. Lasers operating at various infrared wavelengths have been utilized in pain research. Lasers provide advantages as compared to radiant heat sources or/and contact heat or electrical current. These are:
highly controllable and reproducible rate of heating,
heat for some wavelengths can be delivered by optical fiber, and
ease of directing laser energy to specific locations.
non-contact stimulation (compared to contact heat)
nerve fiber type/subtype specific stimulation (compared to electrical current)

One problem with many laser sources is that skin damage occurs before or simultaneously with the feeling of pain. Another problem is that laser pulses may produce double sensations that can induce potentials on one type of fiber by suppressing interaction mechanisms between other nerve fibers, for example in spinal cord. This is most frequently seen for laser pulse duration of more than 100 ms. It is known that lasers operating in the range of 980 nm can produce pain in skin tissues. Photons at this wavelength penetrate to about 3.8 mm through skin tissue (as compared to CO2 laser radiation which penetrate only a few microns). Therefore, typical prior art lasers as well as other radiant contact heat stimulators deposit their heat energy close to the skin surface and are unable to provide selective activation of CMi fibers.

### Nociceptors: A-Delta Fibers and C-Polymodal and Silent, Mechano-Insensitive Fibers

Basic research in pain, migraine, analgesia and pharmacology has been accelerating over last several years. One of the results of this work has been the clear demonstration of the differential involvement of different pain sensing nerve cells (called nociceptors). There are two main classes of pain sensing nociceptors in the skin and other peripheral tissue: myelinated A-delta nociceptors and un-myelinated C nociceptors that are divided to two subtypes: chemo-mechano-heat sensitive polymodal C fibers (CMH) and silent, CMi fibers. Sensations evoked by activation of these two different nociceptor types are quite distinct. A-delta fiber mediated pain is typically described as sharp, or piercing. C polymodal fiber mediated pain, on the other hand, is usually described as burning or aching. The sensation (modality of response) to the stimulation of CMi fibers is still remain unknown, because lack of any selective activation method. There is also a dramatic difference in the latency to pain after activating these two nociceptor types. For example, a rapid pin-prick to a foot can produce two distinct pain sensations: first, a sharp pain which ends when the pin is removed followed by a second, burning sensation which may be felt well after the needle has left the skin. The first pain is mediated by A-delta nociceptors, and the second pain is mediated by C fiber nociceptors. Thus, activation of these two nerve types has a different meaning to the body. Numerous physiological, anatomical, and pharmacological distinctions have also been described as being distinct between these two nerve types. For example, morphine is much more effective in inhibiting C fiber mediated pain than A delta fiber mediated pain. Included among these is the finding that, with a constant stimulus, such as a wound, A-delta nerve cells respond robustly at first, but then rapidly become to quiescent. On the other hand, C fibers, with the same stimulus, continue to fire continuously. A delta fibers are usually responsible for mediating of sharp pin prick pain and C fibers for warmth sensations and hot/burning pain.

Some chronic pain syndromes, e.g., diabetic neuropathic pain and chemotherapy induced may be mediated primarily by CMi fiber spontaneous activations. Microneurographic studies in patients with painful diabetic neuropathy demonstrate clear increases in sensitivity of unmyelinated mechano insensitive (C) nociceptors compared to mechano heat sensitive (C) nociceptors. Thus, the ability to accurately evaluate single CMi or CMH fiber function would be useful in both in diagnosis of these diseases as well as for following patient progress.

What is needed is a product and process for producing controlled selective activation of CMi fibers that causes controlled brief neurogenic flare reaction enable for reproducible stimulation. Specifically, there is a need for the development of a small, portable, commercially viable infrared laser stimulator that will permit mapping and differential assessment of CMi fibers and quantitative measurement of pain signals carried by CMi fibers and associated neurogenic flare (axon reflex flare). Besides, such device will permit controllable over-stimulation of spontaneously active CMi fibers that leads to their de-functionalization (depleting) and could be used for monitoring efficacy of pain and migraine treatment based their affect to threshold, size and kinetic of laser induced neurogenic fare.

### SUMMARY OF THE INVENTION

The present invention provides a process and a portable laser system for pain and migraine research, clinical testing, diagnosis and management. A diode laser is used to produce warmth, tickling, itching, touch, burning/hot pain and pin-prick pain with no tissue damage. The device and methods of the present invention can be used for activation of a CMi sing e nerve fiber, groups of nerve fibers, nerve fibers of single type only as well as inducing of neurogenic flare and its measurement, all with no damage to skin tissue and nerves fiber or especially to de-functionalization (depleting) of CMi fibers with no damage to skin tissue. The present invention is especially useful for research of human or animal sensitivity, pain and migraine management, personalised medicine, drug investigation and testing, and in psychophysiology and electrophysiology studies. The device and methods permit non-contact, reproducible and controlled tests that avoid the risk of skin damage Applicant has shown that tests with human subjects utilizing embodiments of the present invention correlate perfectly with laboratory test with nerve fibers of rats and pigs. The device and the methods can be applied in a wide variety of situations involving the study and treatment of pain and migraine. Preferred embodiments of the present invention provide laser systems and techniques that permit mapping and single mode activation of CMi vs. CMH, C-fibers and monitoring changes of level of CGPR and substance P. The diode laser may be used to activate and deactivate (de-functionalize) heat sensitive nociceptor(s) including: A and C polymodal (CMH) and/or mechano insensitive (CMi) nociceptors, trpv1 positive nerve cells and trpv2 positive nerve cells. The device and process of present invention can be used for investigation and control of central and cortical activation together with functional magneto resonance imaging systems. The device and method can be used to control local and central anesthesia during surgery procedure as a feedback system together with electroencephalography, magnetoencephalography and functional magnetic resonance imaging. The device and method permit non-contact, non-invasive, reproducible activation (stimulation) as well as deactivation or/and blocking an of single nociceptors or groups of nociceptors in vivo and in vitro. The device and method can be used to monitor and predict efficacy of migraine preventive medicine by measurement changes in thresholds of CMi fibers activation and neurogenic flare its duration and intensity. Preferred wavelength ranges is 800 nm to 1600 nm.

The present invention is an important improvement to the to the invention described in U.S. Pat. No. 7,402,167 granted to Applicant. Features of that pkior art patent included: A process for stimulating nerves for conducting nerve research and investigations, said process comprising:
A) generating pulses of infrared light with a diode laser system,
B) controlling said laser to produce laser pulses of desired duration and power to produce a desired pulse power profile,
C) directing at least a portion of said pulses of infrared light to a target comprising a single nerve or a portion of said single nerve so as to produce single mode stimulation of the nerve.
   Improvements described in this patent application include:
A) a diode laser producing laser pulses in a range of 100 ms to 10 sec
B) a TV infrared camera adapted record images of a desired region of skin is used to record neurogenic flare,
C) a thermal camera adapted to monitor a desired region of skin to control laser induce temperature and to record neurogenic flare,
D) a cooling system adapted to cool a desired region of skin, and
E) an LED adapted to illuminate desired regions of skin.

### BRIEF DESCRIPTION OF THE DRAWINGS

FIG. 1 is a block diagram of a preferred embodiment of the present invention.

FIGS. 1A, 1B and 1C show various types of laser delivery systems.

FIGS. 2A and 2B show two additional types of delivery systems.

FIG. 3 shows a light delivery system with thermal imaging temperature feedback loop is similar to FIG.

FIG. 4 shows a two-dimensional scanner to map test locations and to provide treatment of affected painful areas on skin.

FIG. 5 shows two light delivery systems (two laser probes) to directly measure the speed of conduction of nerve fibers.

FIGS. 6A and 6B show a temperature pre-set single laser pulse and sequence of laser pulses similar that were used for stimulation of CMi fibers and neurogenic flare and nerve fiber de-functionalization (depletion) with and without skin cooling.

FIG. 7 A, shows kinetic of neurogenic flare in healthy subject in response to a laser pulse.

FIG. 7 B, shows kinetic of neurogenic flare in healthy subject in response to a laser pulse.

FIG. 7 C, shows kinetic of neurogenic flare in healthy subject in response to a laser pulse before and after CGRP treatment.

FIG. 8 shows the skin surface temperature during laser-induced de-functionalization (with cooling a) by cryogenic gas spray (liquid CO2).

FIG. 9 shows hand-piece schematic view of hand-piece with temperature control and video recording of neurogenic flare: a) without cooling b) and c) with spray and cuvette cooling.

FIG. 10 shows block diagram of hand piece connection to the controller, diode laser and liquid CO2 gas tank.

FIG. 11 is a table providing a description and some alternate names for some of the terms and acronyms used in this application.

### DETAILED DESCRIPTION OF PREFERRED EMBODIMENTS

### Diode Laser System at 980 nm

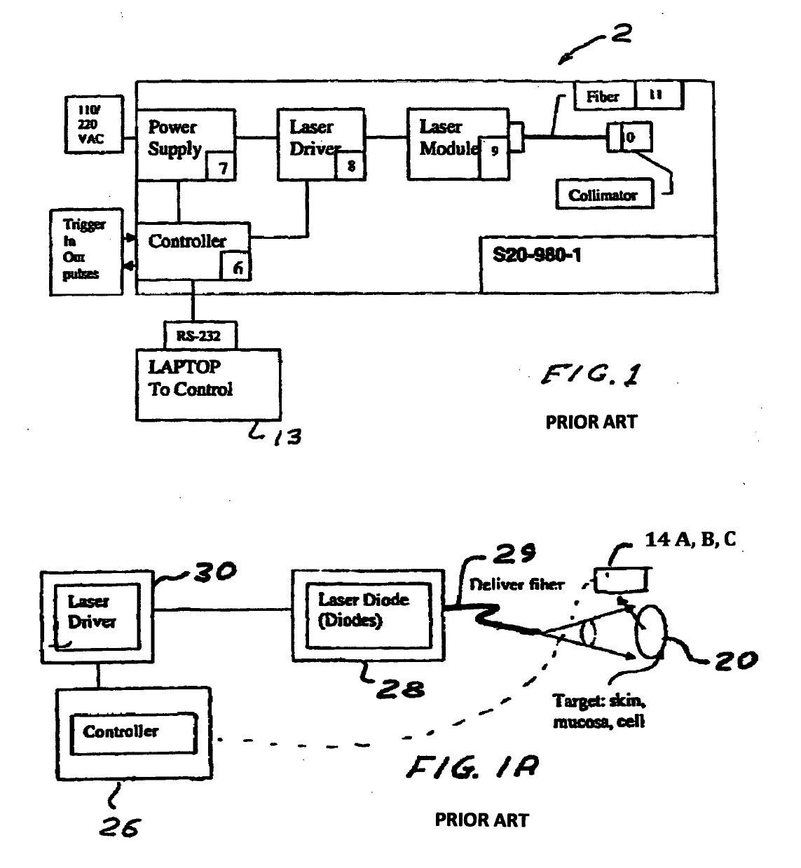

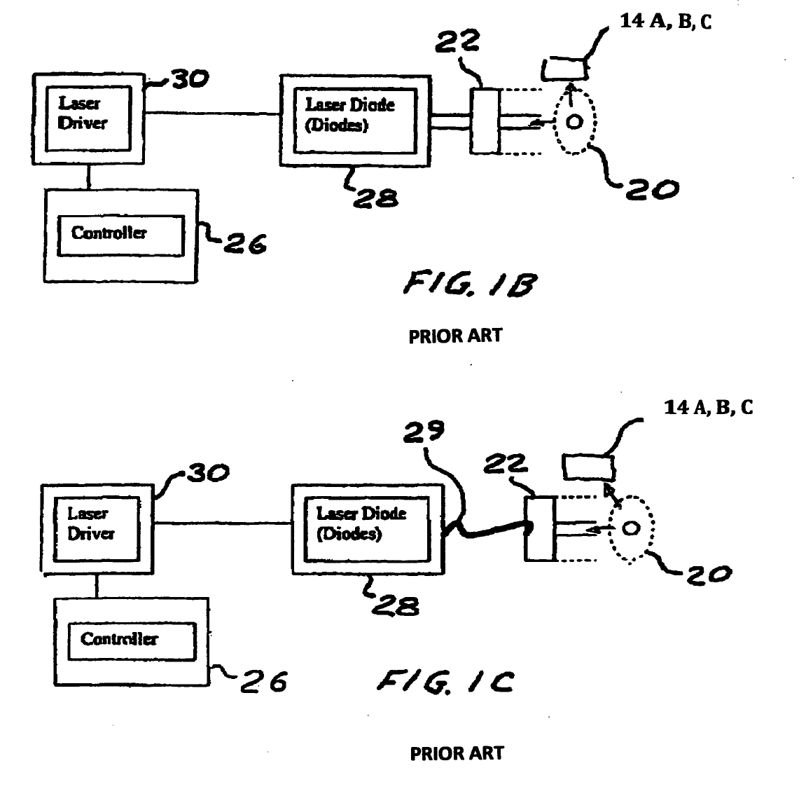

FIG. 1 is a prior art block diagram of a preferred embodiment of the invention described in U.S. Pat. No. 7,402,167. The present invention adds a collimator, a TV camera for recording of laser induced blood perfusion, a thermal camera to control laser induced temperature, an infrared LED for better contrast the blood perfusion, a skin cooling system and an LED.

FIGS. 1A, 1B and 1C are also prior art drawings from U.S. Pat. No. 7,402,167. The present invention also k provides a TV camera for recording of laser induced blood perfusion, a thermal camera to control laser induced temperature, a skin cooling system and an infrared LED for better contrast the blood perfusion.

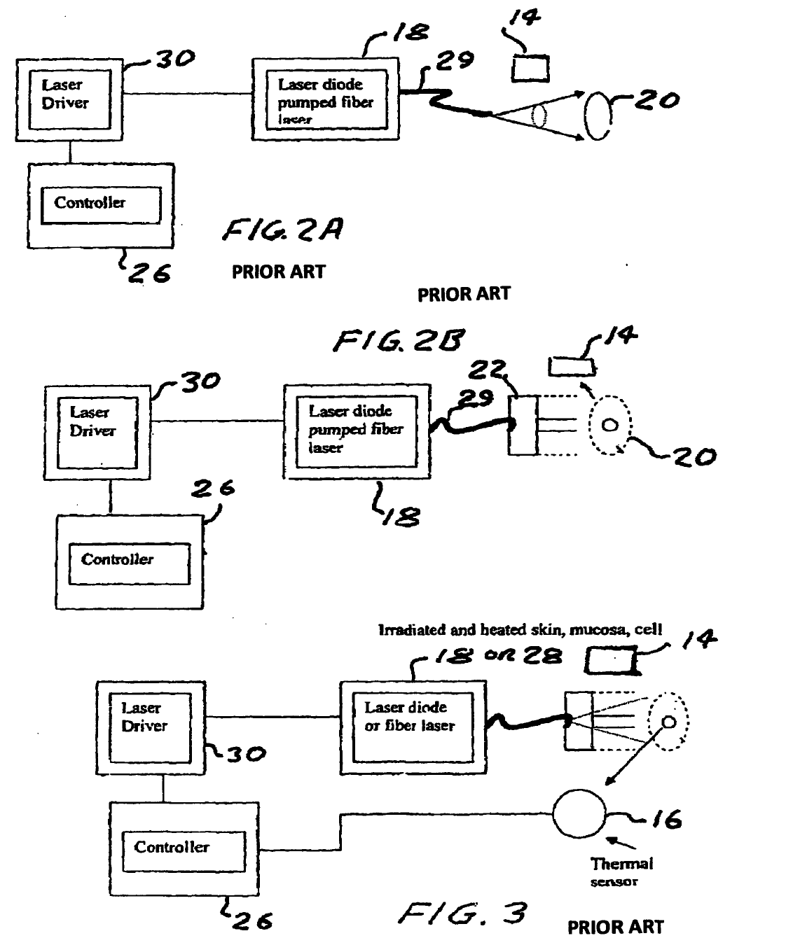

FIGS. 2A and 2B show two additional prior art types of delivery systems from U.S. Pat. No. 7,402,167. The present invention replaces the prior art infrared camera with a TV camera for recording of laser induced blood perfusion, a thermal camera to control laser induced temperature, a skin cooling system and an infrared LED for better contrast the blood perfusion.

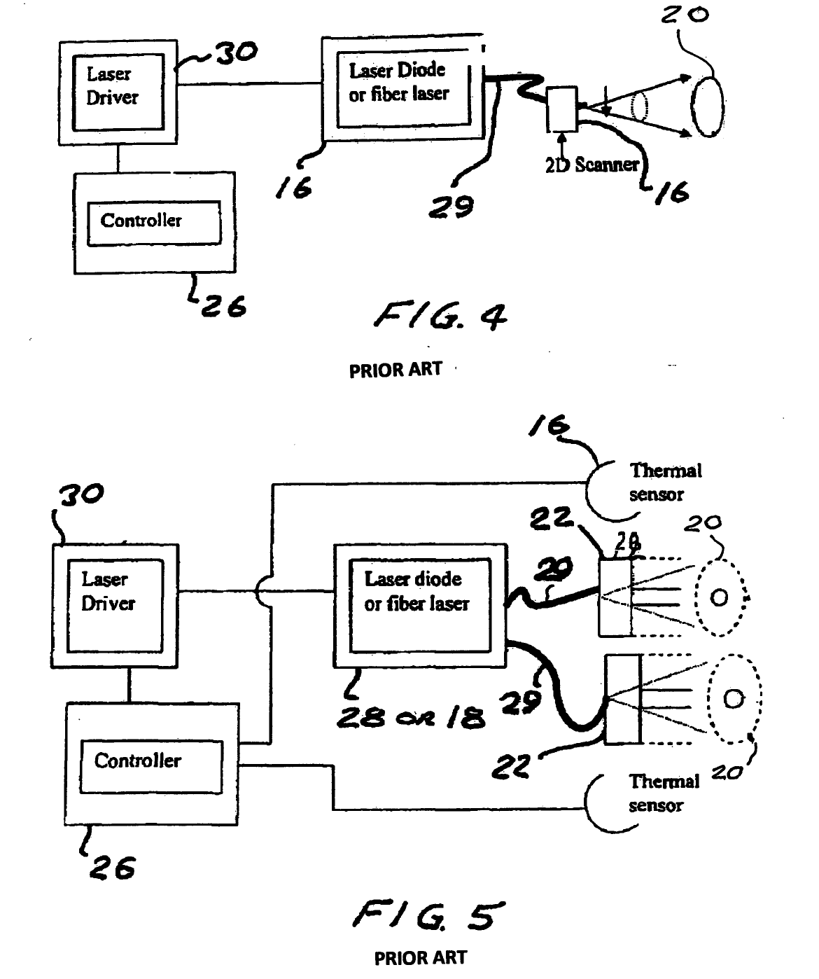

FIG. 3 shows the prior art light delivery system with thermal imaging temperature feedback loop from U.S. Pat. No. 7,402,167. The present invention adds the portable infrared thermal camera, TV camera with polarize filter for recording neurogenic flare (blood perfusion) connected to controller for real time processing, LED for irradiation skin for better contrast of neurogenic flare and skin cooling system based cool gas flow or cooled sapphire for additional skin protection during repeatable stimulation of CMi fibers.

FIG. 4 is also a prior art drawing showing a two-dimensional scanner to map test locations and to provide treatment of affected painful areas on skin from U.S. Pat. No. 7,402,167. As indicated above the present invention adds but with additional components: a TV camera for recording of laser induced blood perfusion, thermal camera to control laser induced temperature, skin cooling system and infrared LED for better contrast the blood perfusion

FIG. 5 again from shows U.S. Pat. No. 7,402,167 two light delivery systems (two laser probes) to directly measure the speed of conduction of nerve fibers but the present invention includes the additional components referred to above

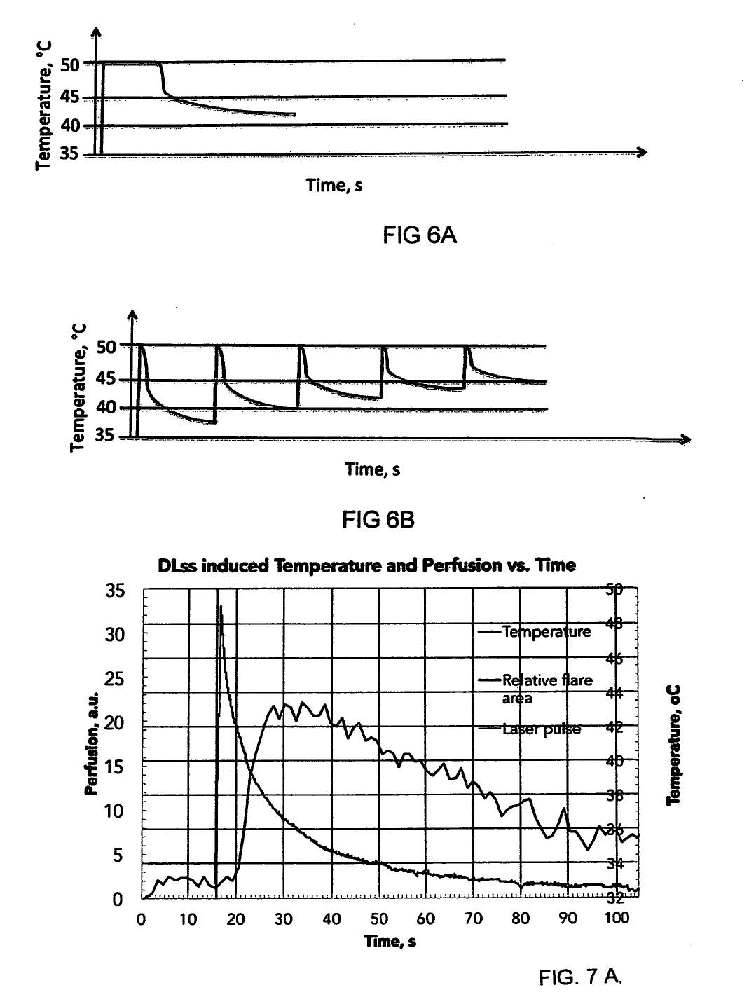

FIGS. 6A and 6B show a temperature pre-set single laser pulse and sequence of laser pulses similar that were used for stimulation of CMi fibers and neurogenic flare and nerve fiber de-functionalization (depletion) with and without skin cooling.

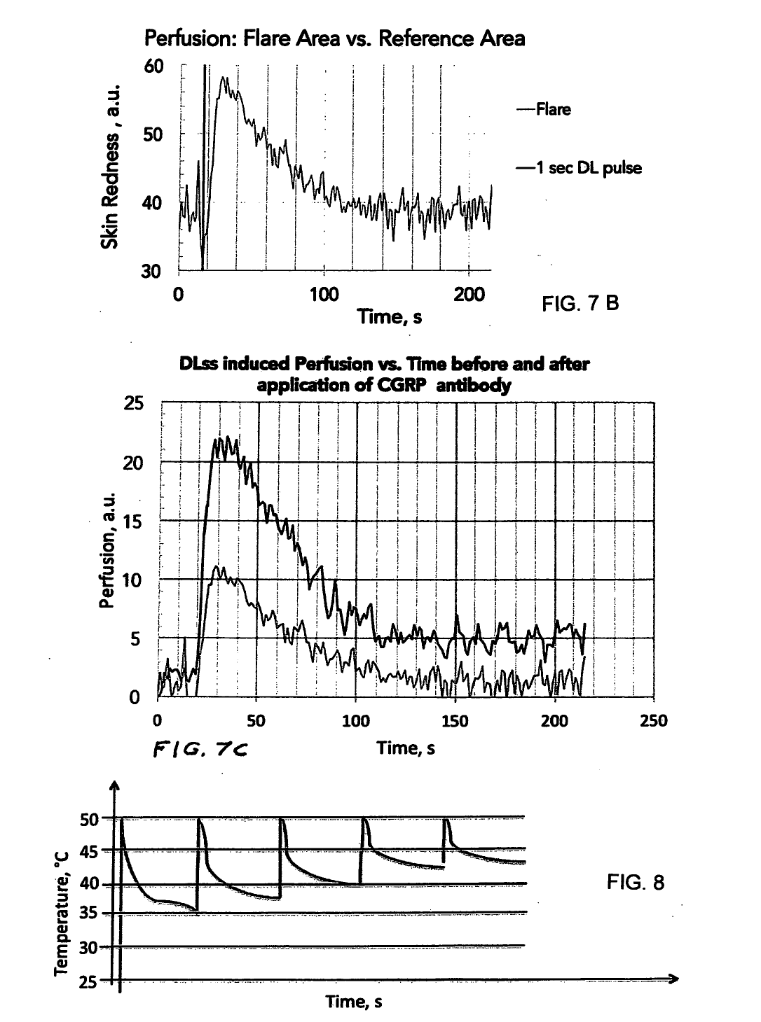

FIG. 7 A, shows kinetic of neurogenic flare in healthy subject in response of laser pulse with peak temperature of 49° C. and duration of 1 s that was not evoked painful sensation. The laser light was applied to skin of inner forearm. Temperature was recorded by thermal camera, the reflected laser light by TV camera and blood perfusion by Speckle Imager (Perimed Corp, Sweden),

FIG. 7 B, shows kinetic of neurogenic flare in healthy subject in response of laser pulse with peak temperature of 49° C. and duration of 1 s that was not evoked painful sensation. The laser light was applied to skin of inner forearm. Temperature was recorded by thermal camera, the reflected laser light by TV camera and neurogenic flare was recorded by TV camera also

FIG. 7C, shows kinetic of neurogenic flare in healthy subject in response of laser pulse with peak temperature of 49° C. and duration of one second before and after application CGRP antibody.

FIG. 8 shows the skin surface temperature during laser-induced de-functionalization (depleting) with cooling a) by cryogenic gas spray (liquid CO₂); The temperature feedback control peak temperature of stimulation below 50° C. and stop lasing if baseline temperature increased over 47° C. The cryogenic spray was used to provide additional safety. The 5 pulses of 500 ms with inter-pulse interval of 3 s were sufficient to provide de-functionalization (depletion). The processed biopsies (PGP 9.5 biomarker) of the treated and non-treated areas confirmed 50% efficacy of nerve fiber depletion.

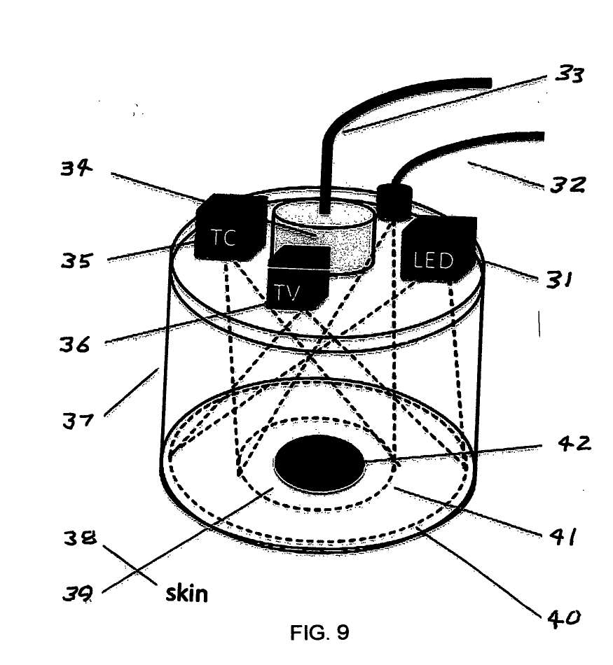

FIG. 9 shows a hand-piece schematic view of hand-piece with temperature control and video recording of neurogenic flare: a) without cooling and with spray and cuvette cooling.

The preferred laser system shown in FIG. 1 is similar to described in FIG. 1 in the U.S. Pat. No. 7,402,167 with additional components: TV camera available turnkey as Model LI-USB30-V034M and from Leopard Imaging Inc. with office located in 1130 Cadillac Court, Milpitas, Calif. 95035, linear polarizer available turnkey for range 600-800 nm from Thorlabs Inc with office located in 56 Sparta Avenue Newton, N.J. 07860, Thermal Vision Camera available turnkey as LI-M38-THERMAL from Leopard Imaging Inc. with office located in 1130 Cadillac Court, Milpitas, Calif. 95035, Infrared Light Emitting Diode available turnkey as Model LED780E from Thorlabs Inc with office located in 56 Sparta Avenue Newton, N.J. 07860.

The laser system includes personal laptop computer (or tablet or specialised microprocessor) 13 programmed to provide the following functions, besides the functions described in the U.S. Pat. No. 7,402,167:
Laser pulse or serial number of pulses that induced pre-set skin temperature from 36 C to 60 C with pulse duration from 100 ms to 60 sec
Processing in real time TV image of blood perfusion

### Advantages of the Present Invention

Some of the uses and advantages that the present invention provides are listed below:
Selective stimulation of mechano insensitive fibers in humans and animal
Behavioral (pain threshold) and instrumental (neurogenic flare threshold) assessment of activation of CMi fibers in animal
Psychophysical (activation and pain thresholds) and instrumental (neurogenic flare threshold) assessment of activation of CMi fibers in humans
Recording of laser induced neurogenic flare
Monitoring of efficacy of medication that control CGRP in humans and animals
Monitoring of efficacy of peripheral painful neuropathy treatment in healthy subjects, patients and animals
Save de-functionalization (depleting) of spontaneously active C fibers that produce pain in pain patients

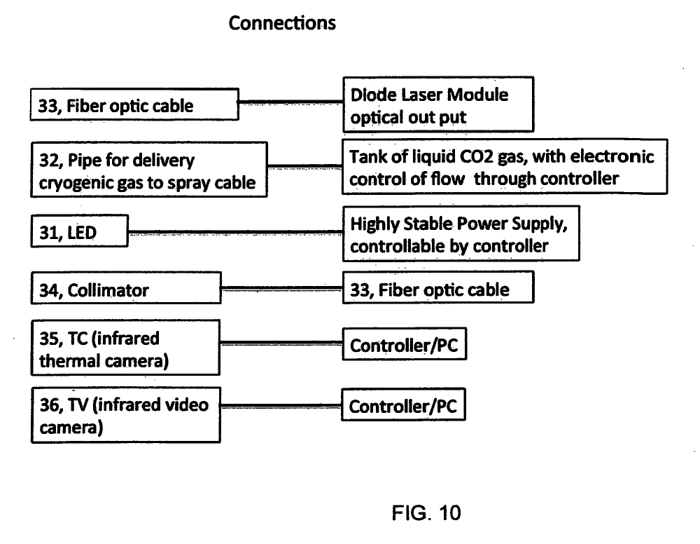

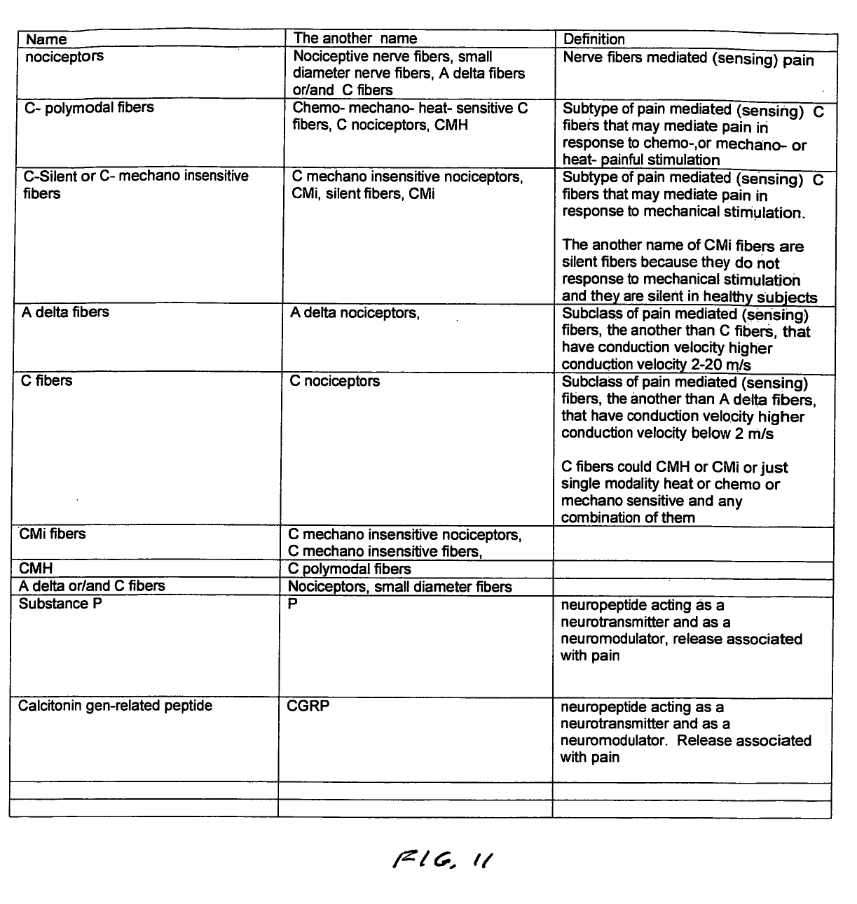

These preferred embodiments are similar as described in the in the U.S. Pat. No. 7,402,167 with addition of models: Lass 10M, Lass M-10M and Lass 11, diode laser fiber type selective stimulators (DLss) from LasMed LLC, 137 Irene Ct., Mountain View, Calif. 94043. Also, the present invention includes additional components: the component infrared camera 14 in the prior art device is replaced by the following components shown in FIG. 9:
1) A TV infrared camera 36 and additionally supplied with polarizer that prevent to observe light scattered from skin surface and increase contrast of observation of changes of blood perfusion. The TV camera is connected to the computer through a controller.
2) A hermal camera 34 that increase an ability control of laser induced temperature and allows monitor profile of heated area compared to thermal sensor described in the U.S. Pat. No. 7,402,167. The Thermal camera provides measurement of max temperature for temperature feedback to pre-set up and control laser induced temperature. It is connected to computer through controller.
3) Cooling system based of application of cryogen spray cooling by CO₂ gas. Preferably the spray device is as described in Majaron paper (Majaron B, Svaasand L O, Aguilar G, Nelson J S. Intermittent cryogen spray cooling for optimal heat extraction during dermatologic laser treatment or a cooled sapphire cuvette similar (as described in Altshuler paper (Altshuler G B, Zenzie H H, Erofeev A V, Smimov M Z, Anderson R R, Dierickx C. Contact cooling of the skin. Phys Med Biol. 1999 April; 44(4):1003-23. PubMed PMID: 10232811) could be used.
4) A 750 nm wavelength light emitting diode with separate power supply to achieve better contrast for blood perfusion recording LED connected to the highly stabilized power supply.
The above components are integrated around collimator as shown on FIGS. 9 and 10.

FIGS. 6A and 6B show the type of laser pulse induced temperature profile that is available with the laser system first described above. FIG. 6A shows temperature vs. time for single pulse and FIG. 6B shows temperature vs time for sequence of 5 pulses. The purpose here is to safely heat the skin to a temperature sufficient for activation of CMi fibers below temperature that may cause skin damage either by peak pulse temperature or build up baseline temperature by thermal feedback described above. The single pulses approach is used for measurement of thresholds of neurogenic flare (activation of CMi fibers) and pulse sequences for nerve fiber de-functionalization (depletion).

FIG. 7A, shows dependence of blood perfusion (kinetic of neurogenic flare) on time after stimulation with non-painful laser pulse that evokes temperature below 50° C. and induces threshold neurogenic flare from forearm skin of healthy subject. Temperature is recorded by thermal camera and the reflected laser light by TV camera as it described above. Blood perfusion is recorded by Speckle Imager (Perimed Corp, Sweden) that is cold standard in the field for the calibration.

FIG. 7B, shows the recording the same process as shown on FIG. 7 A, but TV camera as described above is used to record the neurogenic flare associated redness as well as reflected laser light. The LED and polarizer are used for better contrast as described above. The purpose here is to demonstrate that system of measurement of the neurogenic flare is sufficiently sensitive for determination of threshold of the activation.

FIG. 7C, shows dependence of blood perfusion (kinetic of neurogenic flare) on time after stimulation with non-painful laser pulse that evokes temperature below 50° C. and induces threshold neurogenic flare from forearm skin of healthy subject before and after application CGRP antibody that suppress neurogenic flare. The purpose here is to demonstrate that system allows to monitor efficacy of a CORP treatment.

FIG. 8 shows the skin surface temperature during laser-induced de-functionalization (depleting) with cooling a) by cryogenic gas spray (liquid CO2); The temperature feedback control peak temperature of stimulation below 50° C. and stop lasing if baseline temperature increased over 47° C. The cryogenic spray was used to provide additional safety. The 5 pulses of 500 ms with inter-pulse interval of 3 s were sufficient to provide de-functionalization (depletion). The processed biopsies (PGP 9.5 biomarker) of the treated and non-treated areas confirmed 50% efficacy of nerve fiber depletion. The purpose here is to demonstrate that system may allow temperature safe and efficient to treatment.

FIG. 9 shows example of hand-piece: 37 is the body of hand-piece cylinder made from laser protective polycarbonate (OD 5+ in the spectral range 700 nm to 1000 nm) located on the skin of the subject. On the cap of hand piece are located: 31—LED, 32—flexible pipe for cryogenic gas connected to liquid CO2 trunk, spray, 33 fiber optic connected to 34 collimator, 35 thermal camera connected to power supply and controller, 36 TV camera with polarizer connected to power supply and controller. The internal diameter of hand piece is over 50 mm that provide sufficient area for observation of neurogenic flare. The purpose here is to demonstrate that all components of system for treatment and measurement could be integrated and functioning together to provide research treatment and monitor efficacy of a CGRP treatment.

### Monitoring and Treatment Procedures

### Example 1: Protocol of Selective Activation of C Mechano-Insensitive Fibers in Humans

To the best of Applicant's knowledge, there are not any prior art data in the literature relating to selective activation of CMi as well as activation of neurogenic flare without painful stimulation of CMH fibers. The best, simplest protocol, to assess CMi and activate neurogenic flare is the following:
The best laser set up parameters for lasing of 980
Pulse duration: 500 ms to 2 s,
Beam size: 2 mm to 5 mm
Power: 2-20 W sufficient for laser induced temperature from 45° C. to 55° C.
Density of Energy Range: up to 10 mJ/mm²

The example of practical realization of the combination of pulse duration, beam size and laser induced temperature for reproducible activation CMi as well as short lasting (less than 120 sec) activation of neurogenic flare is shown in Table 1:

TABLE 1
Example of Threshold of activation of CMi fibers
Stimulation of CMi fibers. Irradiated Spot
Diameter 5 mm, Hair Skin

| | | Peak Temperature, |
|---|---|---|
| Pulse, ms | POWER, W, | ° C. |
| 500 | 18 | 56 |
| 1000 | 9 | 51 |
| 2000 | 3.5 | 48 |

The experiment consisted of the following actions:
1) Collimated beam with a diameter of 5 mm, within a range of power of 3.0-10.0 W and a pulse duration of 1 sec is applied to investigated area of skin to determine the individual sensitivity of CMi fibers and to record neurogenic flare. The pulse power is increased from 3 W with step of 0.5 W until first non-painful sensation is evoked. The area of stimulation each time slightly changed to prevent build up of baseline temperature.
2) The found intensity that evoked first non painful stimulation re-applies and possible changes of skin color like a redness caused by neurogenic flare response to laser irradiation is observed and recoded by TV camera with or without of LED lighting is observed in surround area of laser irradiation area of about 30 mm×30 mm.
3) In case of observed redness the laser intensity is decreased with step of 0.1 W until no visible redness is observed and such intensity will be define as threshold of activation of CMi fibers.
4) In case if the intensity that evoked first non painful stimulation will not induce neurogenic flare the laser intensity is increased with step of 0.1 W until the redness will be observed and such intensity will be define as threshold of activation of CMi fibers.

### Example 2 Protocol of Selective Activation of C Mechano-Insensitive Fibers in Humans with Cooled Skin

The steps to define activation threshold of CMi in cooled to skin are similar to the described in the Example 1. The cooled skin mimic to some degree skin and pain sensitivity of patients with peripheral neuropathy who usually develop deficit of pain sensitivity due to decreasing density of pain mediated fibers mainly epidermal C polymodal. The surface cooling also numbs epidermal C polymodal fibers. The only difference is that approximately twice higher laser intensity is required to induce the same temperature that sufficient for activation of CMi fibers. Therefore in step #3 The pulse power is increased from 6 W compared to 3 W in non cooled skin.

### Example 3 Protocol of Selective Activation of C Mechano-Insensitive Fibers in Pigs

TABLE 2
Example of Pain and Activation Thresholds of
CMi fibers
Stimulation of CMi fibers. Irradiated Spot
Diameter 5 mm, duration 6 sec, intensity 2 W Hair
Skin, Pig

| | | Pain Latency | |
|---|---|---|---|
| Pulse Duration, s | Power, W | Response, s | Neurogenic Flare |
| 6 | 2 | 2.5 | Yes, observed |
| | | | before animal |
| | | | reaction to the |
| | | | stimulation* |
| 0.5 | 2 | n/a | No** |
| 1.0 | 2 | n/a | No** |
| 1.5 | 2 | n/a | Yes** |
| 2.0 | 2 | n/a | Yes** |

Notes:
*non-anesthetized;
**anesthetized

The experiment consisted of the following actions:
1) Collimated beam with a diameter of 5 mm, within a range of power of 2 W and a pulse duration of 6 sec is applied to investigated area of skin of non-anesthetized pig to determine the latency that cause a pain response: withdrawal or muscle ripples. The application is stopped then pig response and defined latency of pain threshold is recorded by infrared camera through controller to laptop (see FIGS. 1 and 1 A). The minimum latency for giving power is defined as pain threshold. The stimulation is repeated at least 6 times and all found latency values are averaged. The area of stimulation each time slightly changed to prevent build up of baseline temperature.
2) The animal is anesthetized and intubated. The pulse power is fixed the same as it was used for pain stimulation. The pulse duration decreased to 0.5 s and increased from 0.5 s with step of 0.5 s until first redness caused by neurogenic flare response to laser irradiation is observed and recoded by TV camera with or without of LED lighting is observed in surround area of laser irradiation area of about 30 mm×30 mm. The area of stimulation each time changed to prevent build up of baseline temperature and overlapping of neurogenic flare.

### Example “2” Protocol of Selective Stimulation Single Warmth Sensation or/and Single Hot Pain, Activation of C Fibers

### Warmth Sensation Stimulation

The best laser set up parameters for laser at 980 nm:
Pulse duration: 300 ms to 20 s
Beam size: 3-15 mm
Power: 0.3-10 W
1) A collimated beam with diameter 5 mm, power 1 W and pulse duration 5 sec is applied to investigate area of skin to determine the individual sensitivity of C nociceptors. The lasing is stopped when patient or volunteer report feeling either warmth or hot (burning) pain. The duration of applied pulse is measured. The procedure is repeated 2-3 times and after which the obtained pulse duration is used for the investigation of other areas. Every next pulse is applied to new area of skin if pain or other sensation has not disappeared.
2) The expected pulse duration is between 300 ms and 5 sec. A 300 ms power is increased with step of 0.2 W until the appropriate sensation appears. If the sensation doesn't occurred, then pulses with a duration of 5 sec are applied with increasing of power.
3) The inter stimulation time is at least 20 sec or until the pain sensation has disappeared.
4) For measurement of the Wind Up Effect, the area of stimulation is changed for each successive pulse if pulses applied are separated 2-10 sec.
5) Spatial summation curve: Power density of threshold of warmth or pain vs. Size of irradiated spot. Measurement size of irradiated spot is adjusted to 5 mm, 10 mm and 15 mm and actions 1-2 are repeated for each size with the same selected pulse duration.
6) For measurement of temporal summation curve (Power of Pain Thresholds vs. Pulse Duration) the action 2) is repeated with successive pulses. Pulse power is increased in 0.2 W steps. Expected pulse durations 300 ms-20 sec.
7) The time interval between applied laser pulses has to be more than 3-20 sec to avoid an average heating of skin and skin irritation. Exception is Wind Up Effect where indication of state of skin is subjective rating of pain level and monitoring of surface skin temperature.
8) For determining of the number of pulses that evoked threshold of hot/burning pain in accumulation of heat the square waved pulses within a range of pulse duration of 10-300 ms and inter-stimulus delay of 0.1-3 sec are applied. The examples of repetitive pulse application for C delta stimulation are shown in FIG. 14.
9) In the case of chronic pain syndromes diagnostics, there are differences between normal skin sensitivity and tender areas for example for fibromyalgia syndrome, the actions 5 and 6 are repeated for normal and tender areas before and after treatment.
10) In the case of testing of topical anesthetics or an analgesic drug actions, temperature of surface skin of investigated area at which pain occurs is monitored and data before and after application of topical anesthesia or a analgesic drug is obtained.
11) The pain thresholds as well as tolerance level are individual but suggested actions permit avoidance of temporal skin irritation and skin damage because the pain threshold level for 980 nm is lower than skin damage level.
12) Electrophysiology—Recording Evoked Cortical Potentials: the pulse duration in the range 300-400 ms (up to 2 sec) is selected. The power may be measured as in step 4. The power is adjusted individually in the level between pain threshold and tolerance level. Trigger pulses, synchronized with laser pulse are applied to electroencephalographic protocols. The jitter from pulse with duration of 300 ms-2 sec could not resolve the time delay between even pulses applied to hand and shoulder for a very tall subject. To solve this problem two optical fiber probes method (see FIG. 5) and signal pulse method (see FIGS. 6A,B and FIGS. 15 A, B) were applied, to measure the time delay between two identically stimulated areas. The time delay was measured by delay time between brief trigger pulses.

### Example “3” Application of Protocol Example 2 for Healthy Volunteers

The best laser set up parameters for laser at 980 nm:
Pulse duration: 300 ms to 20 s,
Beam size: 3-15 mm
Power: 1-10 W
Density Energy Range 9-140 mJ/mm²

TABLE 2
Example of threshold Hot Pain and Warmth Stimulations

| | Pulse | Area | | | Energy | Power |
|---|---|---|---|---|---|---|
| Type of | duration, | size, | | Energy, | Density, | Density, |
| Sensation | ms | mm2 | Power, W | mJ | mJ/mm2 | W/mm2 |
| Warmth | 300 | 38.5 | 4.7 | 1410 | 36.6 | 0.122 |
| Warmth | 300 | 176.6 | 5.8 | 1740 | 9.8 | 0.033 |
| Warmth | 1300 | 12.7 | 1.1 | 1430 | 112.6 | 0.087 |
| Hot Pain | 300 | 38.5 | 5.8 | 1740 | 45.2 | 0.151 |
| Hot Pain | 1300 | 19.6 | 2 | 2600 | 132.7 | 0.102 |
| And | | | | | | |
| Warmth | | | | | | |

### Example 4 Group Testing of a Delta Fibers

The following is a description a typical test procedure utilizing the present invention with the application of protocol Example 1 for healthy volunteers and pain patients:

Step 1: Preparing volunteer for test. The volunteers were asked to respond to stimuli after each stimulus was applied, and describing the level and type of evoked sensation, location of sensation, how long the sensation lasts, whether the sensation was single (monomodal) or if more than one sensation were evoked by stimulation.

Step 2: The level of power of laser was adjusted to skin type of volunteer and irradiated spot was selected as close as possible to A-delta receptors.

Collimated beam with diameter 2 mm, within range of power 5.0-10.0 W and pulse duration 100 ms is applied to an investigated area of skin to determine the individual sensitivity and to map the location of A delta nociceptors. The power is increased with step 0.5 W until the first sensation is evoked. After that, the pulse power is fixed and the position of the irradiated spot is scanned (tuned) within an XY frame 5 mm×5 mm to find the location of the nociceptor and pulse power is applied for each new location with spatial steps of around 0.5 mm for fingertips.

The inter-stimulus time was at least 20 sec. The first appeared sensation was rated by the volunteer as barely pricking pain without any other sensations of warmth or hot pain.

### Step 3

After the location of receptors were determined, the summation curve—Power of Pain Threshold vs. Pulse Duration were measured by the following procedure:
1) Collimated beam was adjusted to 1 mm,
2) Duration of pulse set up to 50 ms and pulse power was increased from 2 W until volunteer reported about first appeared sensation.
3) Afterwards, power was increased until volunteer reported that sensation became clearly painful, but decreasing of power of stimulus on 5-10% leads to the disappearance of pain.
4) Afterward, the power threshold was measured for 50 ms pulse duration was increased by step 50 ms and procedure measurement of threshold was repeated. The increment of power was 0.1 W.

### Step 4

The healthy volunteer was tested with pulse durations from 50 ms to 300 ms and beam size of 1 mm. The volunteer reported what around 200-300 ms, he felt on the same level of power firstly warmth and after that pricking pain but for pulses within range 50-150 ms, only single (monomodal) pricking pain was discerned. The sensations were sharp and disappeared in a few seconds after stimulation. The next stimulus was applied not earlier than 20 sec or after the disappearance of painful sensation of previous stimulus.

### Step 5

To measure the tolerance level of pain the level of the power was increased more than threshold level with increment of 0.1 W until volunteer reported that his tolerance level of pain had been reached. The procedure of Step 3 was repeated. The volunteer rated his tolerance level as 10 and his threshold level of pain as 1.

### Step 6

The thresholds of pin prick pain of healthy volunteers were measured in 20 minutes after topical analgesic (capsaicin) was applied on skin. The Steps 2, 3 and 4 were repeated.

### Step 7

To determine a difference in pain threshold and summation curve between healthy volunteer and chronic pain patient the Steps 14 were repeated with patient with chronic hypersensitivity. The result of measurement are shown in Table 3.

### Step 8

The level of skin irritation was measured by repetition of stimulation until redness of skin appeared for skin of healthy volunteers for stimulus duration of 150-300 ms and was recalculated for pulse durations of 50 and 100 ms. The Power level evoking skin irritation is shown Table 3.

As it is shown in Table 3, there is enough room to measure dependence of analgesic action between skin damage power level and initial pain threshold of pin prick pain. The shape of summation curve as well as pain thresholds allow to to determine the hypersensitive area of skin of chronic pain patient and test of selective action (A delta vs C fiber) of analgesic as well as diagnosing of that type of fiber is responsible for conducting of pain of patients.

TABLE 3

| | Power of | Power of | | | Power of Pain |
|---|---|---|---|---|---|
| | Pain | Pain | | Power of Pain | Threshold of |
| | Thresholds | Tolerance | Power of | Thresholds of | Chronic |
| Pulse | of Healthy | of Healthy | Skin | Healthy Volunteer | Pain Patient with |
| Duration, | Volunteer, | Volunteer, | Irritation, | (Capsaicin), | Hypersensitivity, |
| (ms) | (W) | (W) | (W) | (W) | (W) |
| 50 | 9.5 | 11.9 | 16.6 | 14.8 | 7 |
| 100 | 4.5 | 5.6 | 7.9 | 7.0 | 3.5 |
| 150 | 3.2 | 4 | 5.6 | 5 | 2.5 |
| 200 | 2.8 | 3.5 | 4.8 | 4.3 | 2.2 |
| 300 | 2.5 | 3.1 | 4.4 | 3.9 | 2.2 |

### Example 5 Stimulation of C Fibers Comparison of Chronic Pain Patient to Healthy Volunteer

Subjects: A chronic pain patient with hypersensitivity and a healthy volunteer were tested. Both subjects gave informed consent and the experiments were approved by the local institutional review board. The hairy skin of left hand was used for both volunteers. In preparing the subjects testing, they were asked to respond orally immediately after they perceived any threshold sensation and/or to interrupt lasing pulse by pressing STOP button. They then described the level and type of evoked sensation, location of sensation, how long the sensation lasted, whether the sensation was single (monomodal) sensation or if there were more than one evoked sensations by a single stimulation. Afterwards, the threshold pulse was applied 3 times to different areas with interval of ~3 min or after the previous sensation (pain) had disappeared. The appearance redness was an indication of skin irritation.

Step 1: To test duration of pulse that evoked threshold warmth sensation only, the output power was set to 1.5 W, beam size adjusted to 5 mm. The pulse was applied to the skin. When the volunteer reported warmth (pain) sensation the lasing was stopped. The measured threshold pulse durations accordingly were 1300 ms for hot pain for the healthy volunteer, 800 ms hot pain for the healthy volunteer affected topical capsaicin (capsaicin decreases the thresholds of warmth sensation and hot/burning pain) and 910 ms hot pain thresholds for the volunteer with the hypersensitivity. The pulse duration for tolerance for hot pain was 2000 ms. Other studies has shown that redness (skin irritation) only occurs when pulse is extended to 3000 ms.

The laser radiation was stopped by volunteer. The volunteer used the other hand to push the “stop lasing” button when he perceived the sensation. When this button was pushed the command “stop lasing” was activated, duration of applied pulse was measured and it was indicated on the screen of PC connected to the device by RS232 interface.

For the double-checking of reflex time of volunteer, a infrared CCD camera (Sony) instead infrared thermal camera (14) FIGS. 1A-1C was applied to monitor the irradiated skin and reflex time. This method allowed to measure the real reflex time without time of delay related of individual reaction of volunteers. For tested volunteers the pulsed durations accordingly were: 1270, 650, 770 ms. This method also permitted the monitoring of the size of irradiated spots directly by infra red lasing as opposed to use the aiming beam. However, there was not found any differences between the diameter aiming beam and the diameter acting infra red lasing beam of irradiated spot. Accuracy of beam size measurement was +/−0.50 microns.

Step 2: To measure the speed conductivity of C fiber alternatively of two probe method power which evoked hot pain sensation for pulse a duration of 300 ms was determined. This pulse duration is short enough to use it for measurement of speed conductivity by electroencephalographic recording of cortical evoked potentials. The output power was set up 1 W, spot size 7 mm with an increment of power was selected 0.1 W. The volunteer was asked to report the threshold of pain, skin sensation tolerance.

### Example 3. De-Functionalization (Depleting) of Small Diameter Fibers Including CMi Fibers

### Laser Induced Heat Treatment

Heat may cause temporal de-functionalization (depleting) of pain sensing fibers in patients with neuropathic pain that may provide the similar pain relief as application of capsaicin does. Topical capsaicin and other naturally occurring pungent molecules have long been used as analgesics to treat a variety of chronic pain conditions. The analgesic effects of these compounds have been attributed to their ability to desensitize nociceptors into a lasting refractory state up to their temporal de-functionalization and depleting with refractory time up to 8-12 weeks. Development of nonirritant TRPV1 agonists, i.e. ligands that do not evoke action potentials that lead to pain but desensitize TRPV1, has been actively pursued as alternatives to TRPV1 antagonists for pain relief. The capsaicin treatment requires repeated applications other several weeks of small concentration cream (>1.0%) or one time one hour application of high 8% solution or patch. The disadvantage of capsaicin patch application is induced prolong over 48 hours inflammatory pain due 48 hours residual time of capsaicin in the skin.

Laser induced heat pulses with temperature about 50° C. duration of 500 ms with interval of 3 sec with sequences of 5 pulses (50° C.) similar as shown on FIG. 8 were applied to the cooled by cryogenic CO2 spray to about 23° C. thigh area of skin. In 20 hours after treatment part of irradiated area were biopsied with 3 mm punches together with non-treated control and send to lab for estimation of skin damage Simultaneously to the thigh skin of another subject was treated with FDA approved 8% capsaicin patch. None skin damage was observed. In 6 days after treatment the pain sensitivity was tested (as described above in Example 2 of current application). the response from laser treated area demonstrated 20% decrease of pain sensitivity but response from capsaicin was the same as from control area.

In 7 days after treatment additional biopsies from laser and capsaicin treated areas together with control were taken and “blindly” processed with PGP 9.5 biomarker to determine the integral fiber density of the A delta and C fibers in epidermis and dermis. It was found that in capsaicin biopsies the fiber density was about 10 to 20% below control and laser treatment provide 50% to 60% fiber depletion.

This example 3 shows that laser irradiation permits noninvasive deactivation of TRPV1 positive cell, with heat playing an important role. The mechanism of deactivation and depletion of fibers with laser is more effective and less painful compared to the treatment that provided by 8% capsaicin patch. This mechanism can permit the development of pain relief and local anesthesia techniques for human and animal application.

While the present invention has been described in terms of specific embodiments, persons skilled in the art will recognize that many modifications and additions could be made to the specific embodiments without departing from the basic principals of the invention. For example many different wavelengths could be utilised if they the absorption in skin is within the range of about 0.25 cm^{-I} to 10 cm⁻¹. Fiber optics with core diameters in the range of 5 to 100 microns are good for transmitting the laser pulses. Temperature pulse shapes such as the following are good shapes for many experiments:
1. Increasing temperature to set value with arise time of 10-50 ms from baseline skin temperature up to set up level (45 C-60 C) for single non invasive activation of CMi fibers and neurogenic flare
2. Decreasing of intensity of each next stimulation in a sequence of pulses that are applied for de-functionalization (depleting) to prevent thermal skin damage from single pulse stimulation and baseline temperature build up.
3. Control baseline skin temperature during application of sequence of laser pulses below thermal skin damage level by cooling and decreasing of intensity of stimulation.

Therefore, the scope of the invention should be determined by the appended claims and their legal equivalents.

What is claimed is:

1. A process for stimulating nerves for conducting nerve research and investigations, said process comprising:
A) generating pulses of infrared light with a diode laser system,
B) controlling said laser to produce laser pulses of desired duration and power to produce a desired pulse power profile,
C) directing at least a portion of said pulses of infrared light to a target comprising a single nerve or a portion of said single nerve so as to produce single mode stimulation of the nerve;
wherein the diode laser system comprises:
A) a diode laser producing laser pulses in a range of 100 ms to 10 sec
B) a TV infrared camera adapted record images of a desired region of skin is used to record neurogenic flare,
C) a thermal camera adapted to monitor a desired region of skin to control laser induce temperature and to record neurogenic flare,
D) a cooling system adapted to cool a desired region of skin, and
E) an LED adapted to illuminate desired regions of skin.

2. The process as in claim 1 wherein said infrared light is infrared light at wavelengths of about 980 nm.

3. The process as in claim 1 wherein said nerve fibers are C fiber nociceptors.

4. The process as in claim 3 and including a step of identifying the single type of stimulation as warmth stimulation.

5. The process as in claim 3 and including a step of identifying the single type of stimulation as single hot stimulation.

6. The process as in claim 3 wherein said nerve fibers are CMi fiber nociceptors

7. The process as in claim 1 wherein said nerve fibers are A-delta fiber nociceptors.

8. The process as in claim 7 and including a step of identifying the single type of stimulation as prick pin stimulation.

9. The process as in claim 1 wherein said controller comprises a personal computer.

10. The process as in claim 1 and further comprising a step for sensing temperature of said target.

11. The process as in claim 10 wherein said temperature sensor is configured to provide a temperature signal to said controller and said controller is programmed to utilize said temperature to provide feedback control of said laser in order to provide a desired temperature profile at said target.

12. The process as in claim 1 wherein said controller is programmed to provide laser pulsed according to a predetermined pulse energy profile to produce pain but no tissue injury.

13. The process of claim 1 and further comprising the steps of increasing of power for pulse duration 50-150 ms from power level of 0.5 W with step less than 0.2 W with a diameter of irradiation area 0.5-2 mm lead to produce clear monomodal (single) pin prick pain and selective activation of A delta fibers.

14. The process of claim 1 and further comprising the steps of increasing of pulse duration from 0.3 to 20 sec with power level around 1.5 W with a diameter of irradiation area 5 mm-15 mm lead to inducing of clear monomodal hot pain and selective activation of C nociceptors.

15. The process as in claim 1 and including a step of identifying the single type of nerve as a single nerve cell.

16. The process as in claim 1 wherein the said infrared light is directed to said target using an optical fiber with a core diameter chosen from a group of diameters consisting of: 20+/−15 microns, 60+/−15 microns and 100+/−15 microns.

17. The process as in claim 1 wherein said infrared light is infrared light having a wavelength of about 1450 nm.

18. The process as in claim 1 wherein said infrared light is infrared light having a wavelength of about 1850 nm.

19. The process as in claim 1 wherein said infrared light is infrared light having a wavelength of about 810 nm.

20. A diode laser systems for investigating and treating painful neuropathy comprising:
A) a diode laser producing laser pulse in a wavelength range of 800 nm to 1600 nm,
B) a TV infrared camera adapted record images of a desired region of skin,
C) a thermal camera adapted to monitor a desired region of skin,
D) a cooling system adapted to cool a desired region of skin, and
E) an LED adapted to illuminate desired regions of skin,
F) a polarizer to illuminated unwonted reflected light from skin surface.

21. The diode laser system as in claim 20 wherein the wavelength is about 980 nm.

22. The process as in claim 1 wherein the diode laser single pulse is adapted to generate neurogenic flare below pain threshold.

23. The process as in claim 1 wherein the diode laser single pulse is adapted to generate neurogenic flare below pain threshold and if applied relatedly with interval over 200 sec may reproducibly activate neurogenic flare

24. The process as in claim 21 wherein repeatable application the diode laser single pulse with interval below 120 sec may deactivate, de-functianalize and temporary deplete CMi and other Heat sensitive pain mediated nerve fibers (nociceptors) and therefore provide pain relief for patients with peripheral pain.

25. The process as in claim 1 wherein the diode laser wherein the laser system is adapted to induce surface peak temperature in the range of of 45° C. to 60° C. at wavelengths of about 980 nm.
